(19) United States
(12) Plant Patent Application Publication
(10) Pub. No.: US 2005/0283873 P1
(43) Pub. Date: Dec. 22, 2005

(54) X HEUCHERELLA PLANT NAMED 'PARTY TIME'

(50) TIARELLA X HEUCHERRA; HEUCHERA

(75) Inventor: Gary J. Gossett, Portland, OR (US)

Correspondence Address: KLARQUIST SPARKMAN, LLP, 121 SW SALMON STREET, SUITE 1600, PORTLAND, OR, 97204, US

(73) Assignee: Terra Nova Nurseries, Inc.

(21) Appl. No.: 10/871,738

(22) Filed: Jun. 18, 2004

(51) Int. Cl. A01H 5/00

(52) U.S. Cl. PLT/263

(57) ABSTRACT

A new and distinct cultivar of ×Heucherella plant characterized by its abundant warm pink flowers, compact habit, and maple shaped leaves.

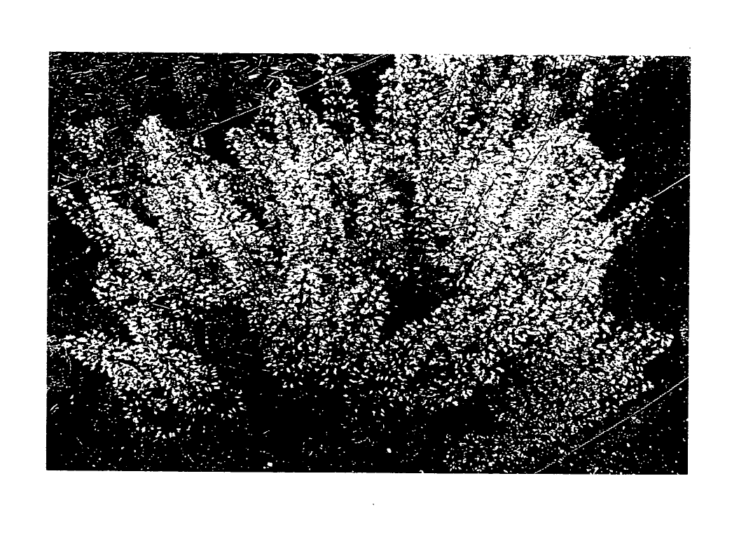

Botanical Designation: ×Heucherella sp.

Variety Denomination: ‘Party Time’

### BACKGROUND OF THE INVENTION

The present invention relates to a new and distinct intergeneric hybrid between Heuchera and Tiarella, given the cultivar name ‘Party Time’. Both genera, Heuchera and Tiarella, are of the family Saxifragaceae. This intergeneric hybrid is known as ×heucherella.

This new cultivar originated from crossing a group of selected Heuchera hybrids with massed pollen from a group of selected Tiarella hybrids. The specific parent plants are unknown.

### SUMMARY OF THE INVENTION

This new Heucherella is distinguished by:

1. Prolific bloom.

2. Warm pink flower color.

3. Compact habit.

4. Maple shaped leaves with a slight white veil, and darkening along the main veins.

5. Excellent vigor.

The new variety has been reproduced only by asexual propagation (division and tissue culture). Each of the progeny exhibits identical characteristics to the original plant. Asexual propagation by division and tissue culture using standard micropropagation techniques with terminal and lateral shoots as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWING

The drawing shows a two-year-old plant of the new variety growing in the spring in the ground in trial fields in Canby, Oreg.

### DETAILED PLANT DESCRIPTION

The following is a detailed description of the new ×Heucherella hybrid based on 1 5 observations of two-year-old specimens grown in the ground in full sun under typical outdoor conditions the trial fields in Canby, Oreg. Canby is Zone 8 on the USDA Hardiness map. Temperatures range from a high of 95 degrees F. in August to 32 degrees F. in January. Normal rainfall in Canby is 42.8 inches per year. The color descriptions are all based on The Royal Horticultural Society Colour Chart.
Botanical name: ×Heucherella.
Cultivar name: ‘Party Time’.
Plant:
   Type.—Herbaceous perennial.
   Form.—Basal rosette.
   Hardiness.—USDA Zone 5.
   Size.—18 cm tall from the ground to the top of the foliage and 35 cm wide.
   Vigor.—Excellent.
   Roots.—Fibrous, freely branching, fine, and white in color. Roots develop easily from cuttings.
Leaf:
   Type.—Simple.
   Arrangement.—Rosette.
   Shape.—Broadly ovate.
   Lobing/division.—5 lobes (+2 small at bottom on some leaves). Lobes are cut ½ way to the midrib, broadly oblong, with the terminal having two secondary lobes.
   Venation.—Palmate.
   Margins.—Crenate.
   Apex.—Mucronulate.
   Base.—Cordate.
   Blade size.—Ranges from 5 to 9 cm long and 5.5 to 8 cm wide.
   Surface texture.—Sparsely pubescent.
   Petiole size.—Ranges from 10 to 14.5 cm long and 2 mm wide.
   Petiole surface.—Pubescent and with glandular hairs.
   Petiole color.—Yellow Green 146C near blade and Greyed Orange 165A near base.
   Leaf color.—Spring leaves —0 Abaxial side —0 Green 138A on and along veins and margins, with a light veil over the rest giving it the Yellow Green 146B cast, and a dark pattern along the main veins toward the leaf base, Greyed Purple 183A. Adaxial side — Yellow Green 147B. Winter leaves — Red Brown, Brown 200B with a silvery veil, Grey 201C. Veins Green 138A.
Inflorescence:
   Type.—Thyrse.
   Number of thryse in first springflush.—75.
   Number of flowers.—Ranges from 65 to 150 per thyrse, on top 17-20 cm of peduncle.
   Peduncle.—With 1 to 5 leaves and flowering branches. Height — 40 cm. Diameter — 0.2 cm. Color — Burgundy, Greyed Purple 183A. Surface — Glandular hairs.
   Pedicel.—Length — 0.5 to 2 cm. Surface — Glandular hairs. Color — Greyed Purple 183A.
   Bloom period.—Early April to late May. Sporadic rebloom until fall in Canby, Oreg.
Flower bud:
   Size.—0.4 cm deep and 0.2 cm wide.
   Description.—Oblong, downfacing, with glandular hairs.
   Color.—Deep pink, Greyed Purple 185C.
Flower:
   Type.—Perfect, sepals petaloid.
   Shape.—Campanulate.
   Size.—0.9 cm wide and 0.6 cm deep.
   Petal count.—5.
   Petal shape.—Oblanceolate with a clawed base, apex acute, margin entire.
   Petal size.—0.5 cm long and 0.1 cm wide.
   Petal color.—Pink, Red Purple 63C on inner and outer surface.
   Petal surface texture.—Satiny.
   Calyx.—Petaloid, divided ⅔ the way to the base, with glandular hairs.
   Calyx shape.—Campanulate with 5 rounded entire lobes, apex acute.
   Calyx size.—0.6 cm wide and 0.4 cm deep.
   Calyx surface texture.—Glandular pubescent.
   Calyx color.—Outside: Red Purple 63D at base darkening toward Red Purple 63B at lobe, with an even darker spot of Greyed Purple 185A at apex. Inside: Same as above.
   Stamen.—5, sterile, filaments of White 55D are 3 mm long terminated with oval, flattened anthers, 0.1 mm wide and 0.15 mm deep, no pollen produced.
   Pistil.—1, 7 mm long, White 55D overall, ovoid and 2-beaked, each beak is 2.5 mm long terminated in a narrow stigma.
   Fragrance.—None
   Lastingness.—A thyrse blooms for about 2 weeks on the plant.
Fruit:
   Type.—Occasional Heuchera like two-beaked capsules are formed.
   Size.—4-7 mm.
   Color.—Yellow Green 150C prior to shriveling and aborting.
Seed: None.
Fertility.—Sterile.
Pest and disease tolerance: No major pest or disease problems. All Heucherella may be damaged by root weevils.

### Comparisons to Similar ×Heucherella

Compared to Heucherella ‘Dayglow Pink’ (U.S. Plant Pat. No. 12,164), this new cultivar has more flowers per peduncle and over a wider area of the peduncle and a different leaf shape, broader and more simply lobed. Heucherella ‘Party Time’ also has a slightly veiled leaf unlike ‘Dayglow Pink’.

1. A new and distinct cultivar of ×Heucherella plant as herein illustrated and described.
